(19) United States
(12) Patent Application Publication
(10) Pub. No.: US 2015/0087967 A1
(43) Pub. Date: Mar. 26, 2015

(54) METHOD AND APPARATUS USING MAGNETIC RESONANCE IMAGING FOR TISSUE PHENOTYPING AND MONITORING

(71) Applicants: Oregon Health & Science University, Portland, OR (US); Brigham and Women's Hospital, Boston, MA (US)

(72) Inventors: Charles S. Springer, Jr., Portland, OR (US); Wei Huang, Lake Oswego, OR (US); Xin Li, Beaverton, OR (US); William D. Rooney, Jr., Lake Oswego, OR (US); Ian J. Tagge, Portland, OR (US); James A. Balschi, Hopkinton, MA (US); Yajie Zhang, Auburndale, MA (US)

(21) Appl. No.: 14/395,785

(22) PCT Filed: Apr. 22, 2013
(86) PCT No.: PCT/US2013/037650

§ 371 Date: Oct. 20, 2014

Related U.S. Application Data
(60) Provisional application No. 61/636,562, filed on Apr. 20, 2012.

(51) Int. Cl. G01R 33/563; A61B 5/00; A61B 5/026; G01R 33/56; G01R 33/565

(52) U.S. Cl. CPC G01R 33/56308; G01R 33/5601; G01R 33/56509; A61B 5/0263; A61B 5/4312; A61B 5/4381
USPC 600/419

(57) ABSTRACT

Provided herein is a Magnetic Resonance Imaging (MRI) technique and, optionally, software, collectively referred to as the “shutter-speed” model, to analyze image data of cancer patients. Embodiments provide a minimally invasive, yet precisely accurate, approach to determining whether tumors are malignant or benign by distinguishing the characteristics of contrast reagent activity in benign and malignant tumors. Exemplary embodiments provide MRI measured biomarkers for tumor malignancy determination and monitoring, effectively eliminating or limiting the false positives suffered by existing MRI techniques while also improving tissue phenotyping and therapeutic intervention monitoring and prediction.

### CROSS REFERENCE TO RELATED APPLICATIONS

The present application claims priority to U.S. Provisional Patent Application No. 61/636,562, filed Apr. 20, 2012, entitled “METHOD AND APPARATUS USING MAGNETIC RESONANCE IMAGING FOR TISSUE PHENOTYPING AND MONITORING,” the entire disclosure of which is incorporated by reference in its entirety.

The present application is also related to U.S. Provisional Patent Application No. 61/171,411, filed Apr. 21, 2009, entitled “DCE-MRI Water Signal Analysis for Improved Cancer Identification,” U.S. Provisional Patent Application No. 61/110,404, filed Oct. 31, 2008, entitled “MRI Biomarker for Cancer Identification,” PCT Patent Application No. PCT/US2009/043201, filed May 7, 2009, entitled “Method and Apparatus Using Magnetic Resonance Imaging for Cancer Identification,” and U.S. patent application Ser. No. 13/125,485, filed Apr. 21, 2011, entitled “Method and Apparatus Using Magnetic Resonance Imaging for Cancer Identification,” the entire disclosures of which are hereby incorporated by reference in their entirety.

### GOVERNMENT INTERESTS

This invention was made with Government support under Grant Nos. RO1-NS40801, RO1-EB00422, RO1 HL-078634, RO1 CA-120861, and UO1 CA-154602 awarded by The National Institutes of Health. The Government has certain rights in the invention.

### TECHNICAL FIELD

Embodiments herein relate to identification of tissue, and, more specifically, to methods and apparatus of using magnetic resonance imaging for tissue identification, phenotyping, and monitoring.

### BACKGROUND

Screening for breast cancer represents one of modern medicine's success stories. However, the continued large fraction of false positives in current diagnostic protocols often leads to biopsy/pathology procedures that cause considerable pain, anxiety, healthcare cost, and possibly increased malignancy risk, but which are potentially avoidable. To address this problem, there have been recent calls for the increased use of magnetic resonance imaging (MRI) in breast screening.

The problems associated with false positive results are not unique to breast cancer screening. Other cancers suffer from large numbers of false positive results, causing significant stress as well as often requiring additional costly and painful procedures to confirm or deny the initial results.

Furthermore, the ability to track cancer growth and predict cancer therapy response via methods that allow for tissue phenotyping and monitoring would be significant. MRI screening that can detect two of the major phenotypic properties of cancers, an angiogenic switch and a metabolic switch, would be a very powerful combination. Detecting tumorigenic transformation crucial to cell metastatic potential would be very valuable for therapeutic monitoring.

### SUMMARY

Disclosed in various embodiments are computer-implemented methods for determining a level of cellular metabolic activity for a region of interest. In various embodiments, the method may include receiving a first set of DCE-MRI time-course data for a region, wherein a contrast reagent is administered prior to imaging, identifying a region of interest from the first set of DCE-MRI time-course data for further analysis, and analyzing the data for the region of interest using computer implemented software to produce a first SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity for the region of interest. In various embodiments, the method also may include receiving a second set of DCE-MRI time-course data for the region of interest, wherein the second set of DCE-MRI time-course data is obtained after the region has been treated, analyzing the second set of DCE-MRI time-course data for the region of interest using computer implemented software to produce a second SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity, and determining the difference between the first SSM Tᵢ value and the second SSM Tᵢ value.

Other embodiments include methods of tissue characterization based on water kinetics. In various embodiments, the method may include receiving DCE-MRI time-course data for a region, wherein a contrast reagent is administered prior to imaging, identifying a region of interest from the DCE-MRI time-course data for further analysis, analyzing the DCE-MRI time-course data for the region of interest using computer implemented software to produce a SM K^{trans} value, wherein the water exchange between cells or blood and interstitial spaces is assumed to be substantially infinitely fast, analyzing the DCE-MRI time-course data for the region of interest using computer implemented software to produce a SSM K^{trans} value, wherein the water exchange between cells or blood and interstitial spaces is assumed to have a finite speed resulting from interaction with the contrast reagent, analyzing the DCE-MRI time-course data for the region of interest using computer implemented software to produce a SSM Tᵢ value that accounts for transcytolemmal exchange effects, and plotting SM K^{trans} and SSM K^{trans} v. SSM Tᵢ to determine a value for the correlation between SM K^{trans} and SSM K^{trans} and SSM Tᵢ.

Also disclosed in various embodiments are computer-implemented methods for determining a level of cellular metabolic activity for a region of interest. In various embodiments, the method may include receiving DCE-MRI time-course data for a region, wherein a contrast reagent is administered prior to imaging, analyzing the DCE-MRI time-course data using computer implemented software to correct for potential ¹H₂O signal reduction due to transverse relaxation effects, identifying a region of interest from the DCE-MRI time-course data for further analysis, and analyzing the DCE-MRI time-course data for the region of interest using computer implemented software to produce a first SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity for the region of interest.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates the pharmacokinetic modeling scheme for DCE-MRI in accordance with various embodiments. The three general compartments for contrast reagent (CR) and for water (blood, interstitium, and parenchymal cytoplasmic) are illustrated, though not in relative proportions to their volume fractions (v_{b}, vₑ, and vᵢ). The pertinent chemical equilibria and their unidirectional rate constants are indicated as well, in accordance with various embodiments.

FIG. 2 illustrates a sagittal, fat-suppressed breast DCE-MRI image (panel A) containing a malignant invasive ductal carcinoma (IDC) tumor (circled contrast-enhanced region-of-interest). Pharmacokinetic K^{trans} parametric maps of the tumor, generated by the Standard Model (FXL-constrained) and two members of the Shutter-Speed Model family (FXR-allowed) and (SXR-allowed), are shown in panels B, C, and D, in accordance with various embodiments.

FIG. 3 illustrates 2D scatter plots of the Standard Model (panel A), and the Shutter-Speed Model (Panel B) (FXR-a) results from Table 1. The ordinates measure the K^{trans} and the abscissae the kₑₚ parameters. The black circles mark the positions for ROIs of lesions that were found by biopsy/pathology to have large malignant fractions, while the triangles are those for lesions found to be solely benign. An outlier (Table 1, patient 5) is plotted in inset panels C and D. Dashed concentric quarter-circles are drawn with radii of 0.19 and 0.23 min⁻¹. The points for two patients (3 and 7) are marked as gray circles with black cores. These represent lesions with only very small malignant fractions, in accordance with various embodiments.

FIG. 4 illustrates a 1D scatter plot derived from Table 1. The ordinate, ΔK^{trans}, is [K^{trans} (SSM)-K^{trans} (SM)]: SSM is FXR-a and SM is FXL-c. The values for the lesion ROIs of all 22 subjects are shown. Those proven malignant are given as filled black circles (these include the two FIG. 3 gray circles with black cores), while those found solely benign are indicated with triangles. The group mean ΔK^{trans} values are indicated with open and filled black squares on the right. Error bars represent (SD) values within each category. One malignant lesion outlier is plotted in an inset, and is excluded from the SD calculation. The horizontal cut-off line drawn at 0.024 min⁻¹ cleanly separates the two lesion groups, all in accordance with various embodiments.

FIG. 5 illustrates how the K^{trans} (volume fraction CR transfer rate constant product, top) and vₑ (extracellular, extravascular space, EES, volume fraction, bottom) fitting results would change if increasing interstitial ¹H₂O T₂* quenching is assumed, in accordance with various embodiments.

FIG. 6, panel A shows a transverse pelvic DCE image slice (anterior up/inferior perspective, approximately 34 seconds post CR injection) of a research subject. Two ROIs are indicated within the prostate gland: one in an area of retrospectively-confirmed prostate cancer, left; and the other in contralateral normal-appearing prostate tissue, right. FIG. 6, panel A also plots the arterial input function obtained from an ROI in a femoral artery. Its magnitude was adjusted using a custom-written numerical approach and an obturator muscle ROI for reference tissue. The time-course from the first-pass was used to estimate blood volume fraction; in accordance with various embodiments, in accordance with various embodiments. Panel B illustrates color-matched tissue data time-courses (points) and representative fittings, in accordance with various embodiments.

FIG. 7 illustrates an article of manufacture, in accordance with various embodiments.

FIGS. 8A and 8B show significant inverse correlations between SM K^{trans} and τᵢ parameters for an entire lesion population (FIG. 8A) and malignant lesions (FIG. 8B), in accordance with various embodiments.

FIG. 9 shows an exemplary example of parametric K^{trans} and τᵢ maps wherein color hot spot maps are overlaid on DCE-MRI images from a malignant (top panel) and a benign (bottom panel) lesion, in accordance with various embodiments.

FIG. 10 shows spatial, heterogeneous distribution of tumor perfusion/permeability (P), hypoxia (H) and necrosis (N) of a representative tumor slice from an experimental tumor, including K^{trans} and τᵢ maps of the tumor slice obtained from SSM analysis, in accordance with various embodiments.

FIG. 11 depicts the spatial distribution of DCE-MRI parameters in well-perfused, hypoxic and necrotic areas of a tumor, including corresponding histograms of the regions with mean (variance) demoted, in accordance with various embodiments.

FIGS. 12A and 12B show examples of tumor region of interest (ROI) parameters K^{trans}, ΔK^{trans}, and τᵢ% changes plotted against relative changes in tumor density (RCTD; FIG. 12A) and residual cancer burden (RCB; FIG. 12B) values for three patients receiving neoadjuvant chemotherapy, in accordance with various embodiments.

FIG. 13 shows SSM K^{trans} and ΔK^{trans} maps of a pathologic partial response (pPR) to chemotherapy and a pathologic complete response (pCR) to chemotherapy, in accordance with various embodiments.

FIG. 14 shows DCE-MRI parameters after completion of neoadjuvant chemotherapy that correlate with residual cancer burden (RCB) and may therefore be indicators of residual disease, in accordance with various embodiments.

FIG. 15 shows SM K^{trans}, SSM K^{trans}, and ΔK^{trans} maps at two time points (before therapy (TP₀) and after two weeks of Sorafenib only treatment (TP₁) of a tumor with 95% necrosis and a tumor with 50% necrosis (both necrosis levels determined at a third time point (TP₂)-8 weeks after TP₂, during which time Sorafenib plus chemotherapy was administered)), in accordance with various embodiments.

FIG. 16 shows a column graph of whole tumor region of interest (ROI) MRI biomarker (parameter) % changes after two weeks of Sorafenib only treatment (TP₁) relative to before therapy (TP₀), in accordance with various embodiments.

FIG. 17 show a scatter plot of % changes after two weeks of Sorafenib only treatment (TP₁) in RECIST (tumor size), ROI ADC (apparent diffusion coefficient), ROI ΔK^{trans}, and histogram median ΔK^{trans} vs. % necrosis at time of surgery (at TP₂), in accordance with various embodiments.

### DETAILED DESCRIPTION OF DISCLOSED EMBODIMENTS

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the present invention, the phrase “A/B” means A or B. For the purposes of the present invention, the phrase “A and/or B” means “(A), (B), or (A and B)”. For the purposes of the present invention, the phrase “at least one of A, B, and C” means “(A), (B), (C), (A and B), (A and C), (B and C), or (A, B and C)”. For the purposes of the present invention, the phrase “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments, methods, apparatuses, and systems using magnetic resonance imaging for cancer identification are provided. In exemplary embodiments, a computing device may be endowed with one or more components of the disclosed apparatuses and/or systems and may be employed to perform one or more methods as disclosed herein.

Embodiments herein provide a Magnetic Resonance Imaging (MRI) technique and optionally newly developed software, collectively referred to as the “shutter-speed” model, to analyze image data of cancer patients. Embodiments provide a minimally invasive, yet precisely accurate, approach to determining whether tumors are malignant or benign. Exemplary embodiments provide MRI measured biomarkers for tumor malignancy determination, effectively solving the false positive riddle from which current MRI techniques suffer.

Although some embodiments throughout are described with reference to breast cancer or prostate cancer, the methods and apparatuses described herein may be utilized for other cancers, such as brain, esophageal, leg osteosarcoma, etc. as well as for any Dynamic-Contrast-Enhanced Magnetic Resonance Imaging (DCE-MRI) analysis where water exchange effects are relevant, including tissue differential/disease state analysis of the brain (Alzheimers, MS, etc.), muscles (such as heart), etc., and quantitative vascular phenotype mapping.

“Quantitative MRI” produces parametric maps of MR, patho-physiological, and/or pharmacokinetic biomarker properties. The DCE-MRI sub-category is particularly significant because it applies to a wide pathology range. In DCE-MRI, the T₁-weighted tissue ¹H₂O MRI signal intensity is acquired before, during, and after the (usually) bolus injection of a hydrophilic, paramagnetic contrast reagent (CR). The CR passage through a tissue region-of-interest (ROI) can cause a transient increase of the longitudinal ¹H₂O relaxation rate constant [R₁≡(T₁)⁻¹] with consequent elevated MR steady-state signal intensity. This elevation may be identified on the MR image.

In DCE-MRI, the neglect of intercompartmental water exchange kinetics considerations can lead to systematic errors in parameters extracted by quantitative analyses. Examples here are the compartmental water mole fractions defining tissue spaces. Therefore, DCE-MRI is also a sub-category of in vivo MR “molecular imaging,” mapping the distribution and/or activity of molecules in living tissues.

In essence, in embodiments, the CR plays the role of the nuclear medicine radioactive tracer. However, in nuclear medicine, the tracer is detected directly (by its radioactivity in disintegrations per second (dps), the amount of tracer present in the tissue, but compartmental localization is not intrinsic to the signal). In contrast, the MRI CR is detected indirectly, via its interaction with water and effect on the nature of tissue ¹H₂O relaxation (so the water interaction with the CR is what is directly traced). Beneficially, the CR is not radioactive. Also, MRI involves no ionizing radiation.

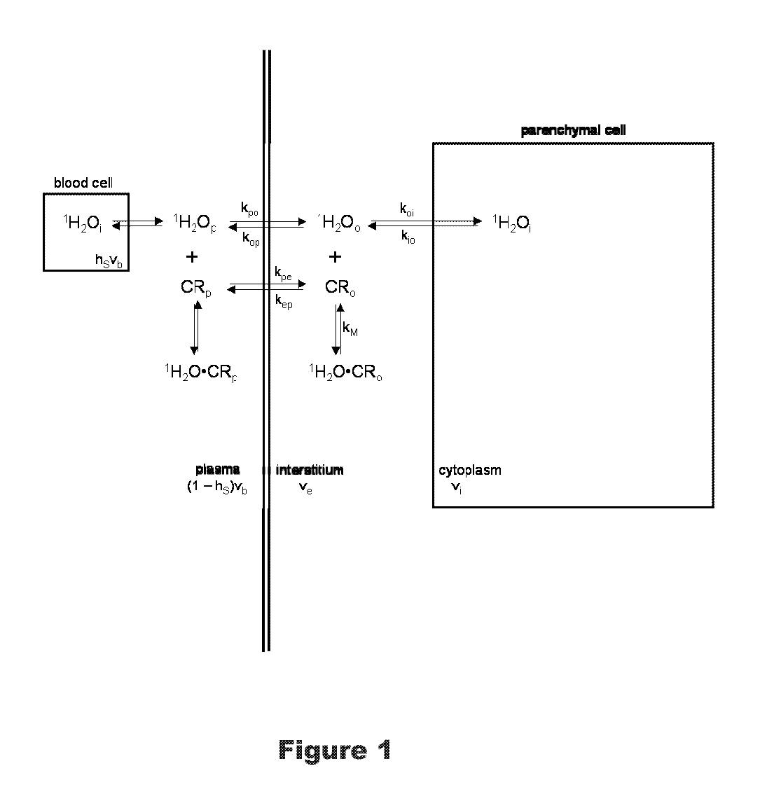

Affecting the recovery of longitudinal ¹H₂O magnetization (e.g., in the magnetic field direction) requires (transient) water CR molecular interaction, as depicted in FIG. 1. The three major loci for tissue water, the cytoplasmae, the interstitium, and the blood, are indicated with subscripts i, o (or e), and b (p, for plasma), respectively. There are water binding equilibria depicted in each compartment in which the CR is thought to enter. The compartmental volume fractions are designated as vᵢ, vₑ, and v_{b}, respectively, though the relative areas in FIG. 1 are not proportional.

The CR and water molecules are never equally distributed in tissue. Therefore, the only way that most water (cytoplasmic) can access CR is via exchange equilibria across cytolemmae and blood vessel walls. These are indicated in FIG. 1 with the unidirectional rate constants, kₒᵢ, kᵢₒ, and kₚₒ, kₒₚ, respectively. In existing methods, tracer pharmacokinetic models are applied directly to MRI data; such methods are referred to herein as the Standard Model (SM). However, this results in the constraint that all inter-compartmental equilibrium water exchange processes be treated as if infinitely fast (kₒᵢ+kᵢₒ→∞, and kₚₒ+kₒₚ→∞). This is not valid, and the assumption may effectively “short circuit” MRI determination of CR compartmentalization, the pharmacokinetic essence. In accordance with embodiments herein, the incorporation of equilibrium water exchange MR effects into pharmacokinetic derivation is referred to herein as the Shutter-Speed Model (SSM). This is accomplished by allowing kₒᵢ+Kᵢₒ and kₚₒ+kₒₚ to be finite.

The SM assumes that water exchange between cells and/or blood and the interstitial spaces is effectively infinitely fast (in the fast exchange limit—FXL). However, when CR is passing through the tumor tissue, the water exchange systems can depart from this fast exchange limit due to the interaction with the CR (and therefore enter into a fast-exchange regime—FXR). This happens for both benign and malignant tumors; however, the exchange difference between FXL and FXR, as far as K^{trans} is concerned, is significantly greater for malignant tumors as opposed to benign tumors. For benign tumors, the exchange difference is typically below 0.025 min⁻¹, whereas for malignant tumors, the exchange difference is typically above 0.025 min⁻¹. This differentiating line provides a threshold against which the obtained values may be compared to classify a tumor or tissue sample. In embodiments, a threshold may be established at an exchange difference of 0.02 to 0.03 min⁻¹.

In an embodiment, the shutter-speed model (SSM) accounts for the FXR (therefore including equilibrium exchange effects when the CR passes through) and thus is better able to pick up the “leaky blood vessel” effect which is common in malignant tumors. At a maximum level of CR in the interstitial space, an interstitial water molecule in a benign lesion may typically encounter a CR molecule an average of 60 times before it enters a cell, whereas in a malignant tumor this may happen 260 times on average (4+ times as often). If that difference is neglected (which the standard model does), then it is sufficient to cause significant K^{trans} (the volume-weighted CR extravasation rate constant) underestimations in malignant tumors. Because K^{trans} values are greater for malignant tissues than for benign tissues, if K^{trans} is underestimated, then it may make a malignant tumor seem benign (false negative) or vice versa a benign tissue appear to be malignant (false positive). The SSM model accounts for this difference, and by using the delta K^{trans} (change in K^{trans}) as well as the K^{trans} to kₑₚ comparison, classification of tumors may be accomplished. In accordance with various embodiments, kₑₚ is the unidirectional CR intravasation rate constant; it is K^{trans} divided by the vₑ (the extracellular, extravascular volume fraction available to the contrast agent molecule). The pharmacokinetic analysis of DCE-MRI data yields K^{trans} and kₑₚ.

In accordance with various embodiments, the difference in K^{trans} returned by SSM, as compared to the Standard Model analyses, offers a very high degree of tumor differentiation (e.g., specificity). It is a measure of the shutter-speed effect, which is disproportionally present and important in malignant tumors, that permits differentiation of benign and malignant tumors.

In analyses of DCE-MRI data from patients with suspicious breast lesions initially ruled positive by institutional screening protocols, the SM K^{trans} values for benign and malignant lesions exhibit considerable overlap. The Shutter-Speed Model (SSM) may allow for finite exchange kinetics thus agreeing with the SM K^{trans} value for each of the benign lesions. However, it reveals that the SM underestimates K^{trans} for each of the malignant tumors in this population. The fact that this phenomenon is unique to malignant tumors allows their discrimination from the benign lesions, as validated by comparison with gold standard pathology analyses of subsequent biopsy tissue samples to which the MRI analyses were blinded. Likewise, the SM overestimates kₑₚ, particularly for the benign tumors. Thus, incorporation of the SSM into the screening protocols may preclude the need for the biopsy/pathology procedures that otherwise would yield benign findings.

Thus, in embodiments, two binary classifiers have been developed:

1. “deltaK^{trans}”—the change in K^{trans}; thresholds may be established with the goal/intention of including all true positives. Thresholds may be established as desired to distinguish/classify the tissues/tumors. In an embodiment, further analysis may be conducted via a secondary mapping algorithm (plot of (K^{trans} vs. kₑₚ) to allow for a second determination with respect to those points that are somewhat unclear or fall below a determined threshold.

2. The use of 2D plots (K^{trans} vs. kₑₚ), where the radius of a circle centered at the origin of the plot may be used as a “binary classifier.” In embodiments, the radius of the circle may be used as a threshold to distinguish benign from malignant tumors. Such a threshold may be established at approximately 0.2 min⁻¹, for example, from 0.19 min⁻¹ to 0.25 min⁻¹.

In embodiments, an MRI examination aided by SSM analysis may provide a clearer diagnosis and may be an intermediate step between a mammographic scan and a biopsy intervention if breast cancer is suspected from both the mammogram and the MRI results. Adding this intermediate diagnostic step may greatly reduce or eliminate the number of unnecessary (and possibly all) biopsy surgeries and also reduce the pain, stress and expense for most patients.

It is important to note that the SSM is a generalization of the SM. That is, the SM is but a special case of the SSM. Thus, if the shutter-speed effect is negligible in any tissue, the program will automatically perform a SM analysis. One can test this by computationally constraining the SSM analysis to a SM form. If there is no difference from the result obtained when the SSM analysis is given free rein, then there is no significant shutter-speed effect for that tissue. In the case of breast tumors, this is the case for the K^{trans} biomarker in only benign lesions. However, there is a shutter-speed effect for the vₑ biomarker in benign breast lesions, and it is about the same size as in malignant tumors.

To test SSM, SSM was employed to analyze MR images of 22 women volunteers who had previously screened positive for breast cancer by mammography and/or clinical examination. The shutter-speed software operates by using a complex mathematical formula to track the passage of injected contrast dye through a tumor area. Contrast dyes are commonly used in medical imaging to increase the visibility of tissue abnormalities.

When viewed through the shutter-speed analysis, the MRI data suggested that only seven of the 22 women actually had malignant tumors. These projections were later shown to be 100 percent accurate after each of the study participants underwent subsequent biopsies for pathology determinations. Typically, 75 percent of mammographically-indicated biopsies yield negative pathology results, meaning that an intermediate step such as an MRI determination could greatly reduce or eliminate the number of unnecessary biopsy surgeries.

This population study has been expanded to include 77 breast tumors (in 74 patients) and, with the mapping provision for one rare type of malignant tumor, maintains 100% specificity.

More specifically, data were obtained with consent from patients with positive mammographic and/or clinical MRI reports from standard, institutional breast cancer workups and protocols. All had MRI contrast-enhanced lesions radiologically classified as BIRADS (Breast Imaging Reporting and Data System) four (γ-4, suspicious) or five (B-5, highly suggestive of malignancy). Emphasizing practicability and robustness, the data are of a rather routine clinical nature (and they were obtained at two different institutions, with two different instruments, CRs, etc.): the two different data acquisitions were not optimized for DCE-MRI. For example, though the spatial resolution is reasonable, the temporal resolution is not optimal. Of particular interest is the fact that the adipose tissue —¹H₂C— MR signal was suppressed in the acquisitions at one institution, while at the other institution it was not.

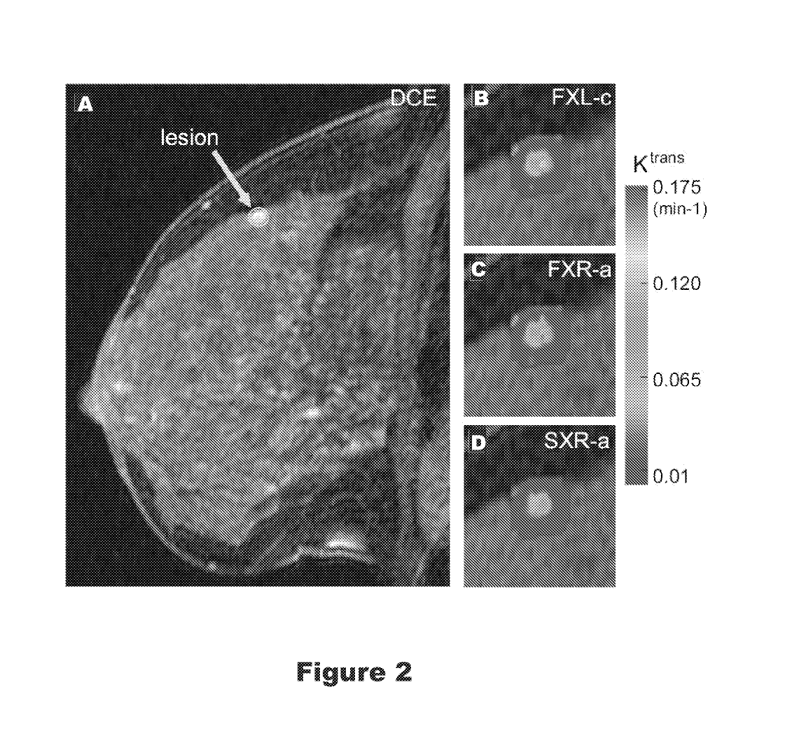

FIG. 2, panel A, shows the DCE pharmacokinetic image of sagittal slice 16 (numbering from lateral to medial) of the left breast of a 52 year-old patient, obtained 2.6 minutes after CR injection. It was acquired with adipose —¹H₂C— suppression (required in the institutional protocol). In contrast to those with no fat suppression, this darker image shows glandular regions brighter than fatty tissue. The ROI circumscribes the enhanced lesion evident in this slice, subsequently found to be a malignant invasive ductal carcinoma (IDC) by pathology analysis. Each of the 22 patients participated in a DCE-MRI acquisition subsequent to her clinical mammography and/or MRI screening but prior to the biopsy procedure and the pathology analysis.

Additional DCE-MRI acquisition details may be found in Li, et al., Dynamic NMR Effects in Breast Cancer Dynamic-Contrast-Enhanced MRI, PNAS, Vol. 105, No. 46, 17937-17942 (2008) (and Supporting Online Material), all of which is incorporated by reference in their entirety. For each of the 22 subjects, ROI DCE-MRI time-course data were analyzed from one sagittal image slice (out of 16 to 40 per breast) that exhibited a lesion to be subsequently biopsied. An ROI boundary was manually drawn around the entire lesion in a pharmacokinetic image showing near maximal enhancement (as in Panel B of FIG. 2). The patients are enumerated in Table 1, below. The FIG. 2 images are from patient 3. The DCE-MRI time-courses were each analyzed with several pharmacokinetic models.

TABLE 1

| | | K^{trans} (min⁻¹) | | kₑₚ (min⁻¹) | | |
|---|---|---|---|---|---|---|
| Patient | BI- | SM | SSM | SM | SSM | |
| Number | RADS | (FXL-c) | (FXR-a) | (FXL-c) | (FXR-a) | Pathology Report |
| 1 | B-4 | 0.073 | 0.147 | 0.389 | 0.249 | DCIS, intermediate nuclear grade |
| 2 | B-4 | 0.110 | 0.180 | 0.452 | 0.447 | IDC, histologic grade II/III; DCIS, |
| | | | | | | intermediate nuclear grade; |
| | | | | | | DCIS ≦25% of total numor mass. |
| 3 | B-4 | 0.087 | 0.131 | 0.161 | 0.147 | IDC present at the edge of the core |
| 4 | B-5 | 0.164 | 0.254 | 0.532 | 0.432 | IDC, histologic grade II/III; DCIS, |
| | | (±0.028) | (±0.029) | | | intermediate nuclear grade; DCIS |
| | | | | | | >25% of total tumor mass. |
| 5 | B-5 | 0.559 | 1.63 | 1.795 | 2.966 | IDC, histologic grade II/III |
| | | (±0.040) | (±0.06) | | | |
| 6 | B-5 | 0.145 | 0.185 | 0.506 | 0.308 | IDC |
| | | | (±0.020) | | | |
| 7 | B-4 | 0.051 | 0.081 | 0.269 | 0.202 | IDC, nuclear grade II, LCIS: |
| | | | | | | moderately differentiated IDC |
| | | | | | | embedded within a lamer benign |
| | | | | | | LCIS |
| 8 | B-5 | 0.033 | 0.034 | 0.106 | 0.053 | LCIS, SF |
| | | (±0.065) | (±0.006) | | | |
| 9 | B-4 | 0.022 | 0.023 | 0.147 | 0.047 | FC |
| 10 | B-4 | 0.051 | 0.058 | 0.306 | 0.155 | FC |
| 11 | B-4 | 0.040 | 0.055 | 0.280 | 0.120 | FC |
| 12 | B-4 | 0.062 | 0.077 | 0.397 | 0.188 | Sclerosed papillary lesion, LCIS |
| 13 | B-4 | 0.027 | 0.028 | 0.048 | 0.039 | FC |
| 14 | B-4 | 0.030 | 0.034 | 0.229 | 0.096 | ADH, SF |
| 15 | B-4 | 0.091 | 0.099 | 0.109 | 0.131 | LCIS, SF, FC |
| 16 | B-4 | 0.078 | 0.087 | 0.188 | 0.130 | LCIS, ADH |
| 17 | B-4 | 0.108 | 0.125 | 0.289 | 0.166 | duct ecrasia, ADH |
| 18 | B-4 | 0.060 | 0.066 | 0.133 | 0.090 | SF, sclerosing adenosis |
| 19 | B-4 | 0.048 | 0.050 | 0.185 | 0.086 | FA |
| 20 | B-4 | 0.026 | 0.028 | 0.174 | 0.066 | FA |
| | | (±0.010) | (±0.005) | | | |
| 21 | B-5 | 0.020 | 0.022 | 0.307 | 0.136 | FA |
| 22 | B-4 | 0.016 | 0.016 | 0.436 | 0.078 | FA |

IDC: invasive ductal carcinoma;
DCIS: ductal carcinoma in situ;
LCIS: lobular carcinoma in situ;
SF: stromal fibrosis;
FC: fibrocystic changes;
ADH: atypical ductal hyperplasia;
FA: fibroadenoma.

For the patients/results presented in Table 1, ROI boundaries around each lesion were separately drawn by each of two independent investigators who were blinded to the pathology results. The analyses of these ROI data were also conducted independently by two investigators. The algebraic means of the model parameters returned from each investigator's fitting were computed lesion-by-lesion.

Each of the fittings neglects the small blood water proton signal (¹H₂O_{b})—thus, these are “first generation” versions. For this situation, the MR exchange system of interest is that for equilibrium transcytolemmal water interchange (kₒᵢ and kᵢₒ, FIG. 1). The system's condition is given by the comparison of the equilibrium kinetics, k=kₒᵢ+Kᵢₒ, with the pertinent MR shutter-speed, T⁻¹≡|R₁ₒ−R₁ᵢ|, where R₁ₒ and R₁ᵢ are the relaxation rate constants for the ¹H₂Oₒ and ¹H₂Oᵢ signals in the absence of exchange. Before CR arrival, R₁ₒ≈R₁ᵢ and T⁻¹<<k. Though k is finite, and invariant throughout the DCE-MRI study, the system is in the fast-exchange-limit (FXL): the kinetics appear infinitely fast, and the measured tissue ¹H₂O R₁ is single-valued. As stated above, the Standard Model assumes that the system remains in the FXL throughout the CR bolus passage, so it is referred to also as the FXL-constrained (FXL-c) model (see FIG. 2, panel B). However, as the CRₒ concentration increases, R₁ₒ becomes increasingly larger than R₁ᵢ and T⁻¹ at least approaches the constant k value. For some period, the measured R₁ remains effectively single-valued, and this has been defined to be the fast-exchange-regime (FXR). Admitting departure from the FXL for the FXR may be referred to as FXR-allowed (FXR-a) (see FIG. 2, panel C). Further CRₒ increase may lead to the condition where R₁ is effectively double-valued: this is referred to as the slow-exchange-regime (SXR). Admitting this is referred to as SXR-allowed (SXR-a) (see FIG. 2, panel D).

For the cases here, the results of FXL-c and FXR-a analyses are presented in Table 1. Careful analyses with the SXR-a model indicate that it is incompatible with these data; an example will be seen below. There are a number of potentially variable parameters. For the SM (FXL-c) analyses, the variables were K^{trans} and vₑ, while for the SSM (FXR-a) analyses, τᵢ was also varied. In terms of the FIG. 1 notation, K^{trans}=vₑkₑₚ=v_{b}kₚₑ, and τᵢ=kᵢₒ⁻¹. The values returned for K^{trans}, a measure of the rate of passive CR transfer across the vessel wall, and kₑₚ, the unidirectional rate constant for CR intravasation (FIG. 1) are given in Table 1. Sample standard deviation measures of parameter uncertainty from individual fittings are given for some entries. These were determined by multiple Monte Carlo fitting calculations. The K^{trans} and kₑₚ values for the malignant tumors (top seven entries) are larger than those for the benign lesions.

Table 1 indicates that the SM does not completely separate the malignant tumors (top seven entries) from the benign lesions with either the K^{trans} or kₑₚ parameters. However, the SSM significantly increases K^{trans} for every one of the malignant lesions, and for none of the benign tumors, as compared to the SM. Furthermore, though the SSM reduces kₑₚ for both malignant and benign lesions, it does this more for the benign tumors. In embodiments, these changes allow discrimination between the SSM and SM results.

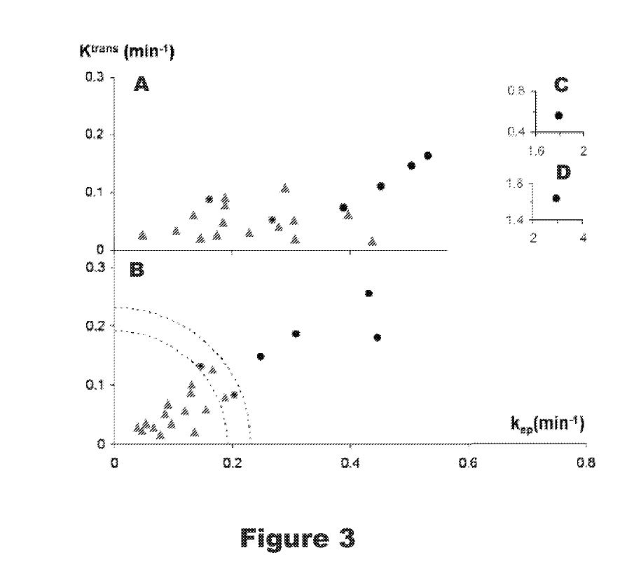

Though neither of the parameters allows the construction of perfect ROC (Receiver Operator Characteristic) plots, the SSM K^{trans} and kₑₚ quantities come very close. These aspects may be seen in the 2D parametric scatter plots of the K^{trans} (ordinate) and kₑₚ (abscissa) values presented in FIG. 3. The ROI values for lesions found by pathology analyses (Table 1) to be solely benign are indicated with triangles, while those with major malignant regions are shown as black circles. The two gray circles with black cores also represent malignant tumors and are discussed below. The results from the SM (FXL-c) analyses are seen in panel A, while those from the SSM (FXR-a) determinations are shown in Panel B. The values for patient 5 are so large that they are shown in inset Panel C and inset Panel D.

In comparing Panel B with Panel A, one can note especially the upward movement (increasing K^{trans}) of the circles and the leftward movement (decreasing kₑₚ) of the triangles, in going from the SM to the SSM. This allows the almost complete separation of these points in Panel B, which is not achieved in any single dimension of either panel. It is important to note that two of the triangles represent B-5 lesions (Table 1): e.g., they were “highly suggestive” false positives. Retaining 100% sensitivity (not missing any malignant tumor), the PPV values for the SM K^{trans}, SM kₑₚ, SSM K^{trans}, and SSM kₑₚ dimensions are: 54%, 39%, 70%, and 70%, respectively. In the Panel B SSM 2D plot, one can draw a dashed quarter-circle of radius 0.19 min⁻¹, that also allows a 78% PPV.

Furthermore, consider the annular region between this and the other concentric quarter-circle, of radius 0.23 min⁻¹. The only two malignant tumors (circles with dark cores within) are those of patients 3 (upper) and 7 (lower). These are cases where the malignant areas are quite small compared with the total tumor area visualized in the biopsy specimen (Table 1). This means that the analyses of whole-tumor ROI-averaged data cause a partial volume dilution of the DCE-MRI parametric values. This can be seen clearly in FIG. 2, Panels B and C, which present K^{trans} parametric maps of the lesion of patient 3. In the SM (FXL-c) and SSM (FXR-a) maps (FIG. 2, Panels B and C, respectively), a clear “hot spot” is seen on the posterior lesion edge. The hot spot has K^{trans} values above 0.16 min⁻¹ in the FXR-a map, considerably elevated above the ROI-averaged magnitude (Table 1).

The hot regions of all seven malignant tumors in this population have SSM K^{trans} values exceeding 0.1 min⁻¹. Except for that of patient 17 (upper triangle in FIG. 3, Panel B annulus, and which uniquely exhibits ductal dilation (Table 1)), this exceeds the ROI-averaged SSM K^{trans} values of any of the fifteen benign lesions. With FIG. 2, Panels B, C, and D, parametric maps of four of the seven malignant tumors are presented. Some hot spots may be as small as 2 mm in diameter. In another indication of potential staging power, a plot (not shown) of “hotness” vs. area of the SSM K^{trans} hot spots in the malignant tumors of patients 5, 6, and 7 demonstrates that these two independently measured quantities are very highly positively correlated. The fact that the SXR-a K^{trans} map of the patient 3 lesion (FIG. 2, Panel D) does not show increased values relative to the FXL-c map (FIG. 2, Panel B), and in fact obliterates the hot spot, is an example of the SXR-a model incompatibility with these data.

The K^{trans} and kₑₚ values are rather well correlated in FIG. 3, particularly in Panel B. The positions of the Panel A and Panel B insets are placed with constant coordinate aspect ratios. Thus, one can visually include the inset points in the correlations. The slope of a line drawn through the points represents the mean vₑ value of these lesions. Such a line for Panel B has a slope near 0.5.

These results indicate a breast cancer screening protocol in accordance with various embodiments herein. The first step of such a protocol may be a clinical examination and/or mammography. A positive result (B-4 or B-5), or suspicion of a mammographically occult lesion, may occasion referral for diagnostic MRI that includes DCE. The radiologist may circumscribe an ROI from the DCE image showing the greatest enhancement. Alternatively, this may be automated (e.g., using Jim 4.0 software; Xinapse Systems; Thorpe Waterville, UK). The computer may very quickly (few seconds) conduct SM and SSM analyses on the mean ROI signal time-course data and produce SSM K^{trans} and kₑₚ values, which can be compared with 2D scatter plots such as those in Panel B. If a patient's point turns out to be in the annulus between the quarter-circles in Panel B, the radiologist may proceed to read K^{trans} parametric lesion maps made from the same DCR-MRI data, though these require more computational time. Hot spots above 0.1 min⁻¹ may be very suspicious for malignancy.

Some oncologists advocate a separate regimen for a malignant ductal carcinoma in situ (DCIS) tumor, possibly simply following it instead of immediate surgery, while others urge excision. The only solely DCIS case in the discussed patient population is that of patient 1. Her position in Panel B is the black point closest to the outer quarter-circle. In fact, another concentric quarter-circle of radius 0.3 min⁻¹ would isolate this point. Its position may be “followed” or tracked over a period of time to see if it moves up and to the right. Inside the inner quarter-circle, most of the benign LCIS lesions are found in the upper right sector, while all of the FA lesions are found near the bottom.

In the analyses so far, pseudo-absolute parameter values have been employed. The SSM success suggests that neglect of equilibrium transcytolemmal water exchange effects may constitute the most significant systematic error in Standard Model DCE-MRI pharmacokinetic analyses.

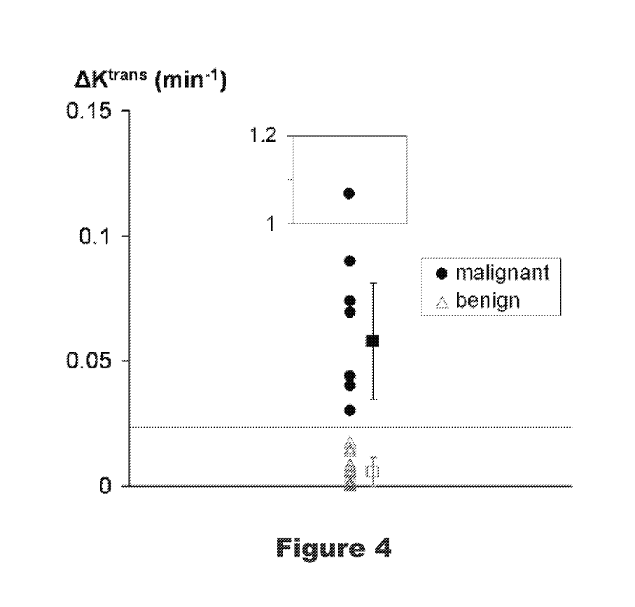

For screening purposes, the most striking aspect of the Table 1 and FIG. 3 results may be that every one of the malignant tumor ROI K^{trans} values (dark circles) is clearly decreased by the SM analysis, while every one of the benign lesion ROI values (triangles) is not. This may be seen even more clearly in FIG. 4, which presents the 1D scatter plot for ΔK^{trans} [≡K^{trans}(SSM)−K^{trans}(SM)]. There is a wide gap between all seven of the dark circles [group mean, 0.06 min⁻¹ (excluding the inset point)], and all 15 of the triangles. The latter set clusters very near zero [group mean, 0.006 min⁻¹]. A clean cut-off line is drawn at 0.024 min⁻¹. Since the only difference between these two models is the allowance for the effect on the NMR signal of finite equilibrium transcytolemmal water exchange kinetics, the NMR shutter-speed effect, this suggests that it is significant (for the K^{trans} magnitude) with the capillary wall permeability obtained for the vascular beds of only malignant breast tumors. Thus, this is very encouraging that analyses of DCE-MRI ROI data first with one pharmacokinetic model and then with the other (which is still accomplished in only seconds) can lead to extremely high specificity in cancer screening. Here, the positive criterion of ΔK^{trans}>0.025 min⁻¹ yields 100% PPV.

Apparently, in the vascular beds of malignant breast tumors only, the interstitial (“outside”) CR concentration, (CRₒ), transiently rises to sufficient values during the bolus passage and the equilibrium transcytolemmal water exchange system transiently departs the FXL to sufficient extent and/or for sufficient duration to substantially invalidate the SM K^{trans} determination. The SSM interpretation is that, during the bolus passage through malignant lesions, the relaxographic T⁻¹ value for the transcytolemmal water exchange process, |R₁ₒ−R₁ᵢ|, transiently approaches or exceeds that for the unchanging exchange rate constant, kᵢₒ+kₒᵢ, (in vivo studies are isothermal) sufficiently for the system to enter at least the fast-exchange regime (FXR), but probably not also the slow-exchange-regime (SXR). R₁ₒ increases with CRₒ, while R₁ᵢ remains constant. This is a manifestation of the varying equilibrium competition for interstitial water molecules between diamagnetic cytoplasmic spaces and paramagnetic interstitial CR molecules (FIG. 1). Informative estimates can be made by comparison of the Table 1 patients 8/4 benign/malignant lesion pair, with SSM K^{trans} 0.034 and 0.254 min⁻¹, respectively. For one of the SSM (FXR-a) fittings of each, the (vₑ, τᵢ) parameters returned are similar: (0.60, 0.40 s), and (0.69, 0.39 s) for benign and malignant, respectively. Thus, the unidirectional rate constants for water cellular entry [kₒᵢ≈(vₑ⁻¹−1)τᵢ⁻¹] are similar (1.7 and 1.2 s⁻¹, respectively), constant, and not infinitely large.

However, before the arrival of interstitial CRₒ, the transcytolemmal water exchange appears infinitely fast in the NMR signal because T⁻¹ is almost negligible. The interstitial water molecules encounter no paramagnetic CRₒ molecules before entering a diamagnetic cytoplasm. However, as CRₒ increases, the rate constant for interstitial water CR encounter, [(CRₒ)/(H₂Oₒ)]τ_{M}⁻¹, also increases [τ_{M}⁻¹=k_{M} in FIG. 1]. While, for the benign lesion CRₒ maximizes at 0.52 mM (at ˜7.5 minutes), this is 1.6 mM (at ˜3.5 minutes) for the malignant tumor. Thus, [(CRₒ)max/(H₂Oₒ)]τ_{M}⁻¹ values are 104 and 313 s⁻¹ for the benign and malignant lesions, respectively. The interstitial water concentration (H₂Oₒ) was 50 M and the mean water lifetime on the CR, τ_{M}, was 10⁻⁷ s. At maximum CRₒ, an interstitial water molecule in the benign lesion encounters a paramagnetic CR molecule on average 60 times (1041.7) before it enters a diamagnetic cell; sufficient, apparently, for the SM 40% vₑ underestimation. While in the malignant tumor, this happens 260 times (313/1.2) on average; more than four times as often. This is sufficient to cause significant K^{trans} underestimations if it is neglected.

Further details regarding the materials and methods used with respect to various embodiments described herein as well as details regarding some of the MRI data acquisitions and analyses may be found in Li, et al., Dynamic NMR Effects in Breast Cancer Dynamic-Contrast-Enhanced MRI, PNAS, Vol. 105, No. 46, 17937-17942 (2008) (and Supporting Online Material); Huang, et al., The MR Shutter-Speed Discriminates Vascular Properties of Malignant and Benign Breast Tumors In Vivo, PNAS, Vol. 105, No. 46, 17943-17948 (2008); Li, et al., Shutter-Speed Analysis of Contrast Reagent Bolus-Tracking Data: Preliminary Observations in Benign and Malignant Breast Disease, Magn. Reson. Med., 53:724-729 (2005); and Yankeelov, et al., Evidence for Shutter-Speed Variation in CR Bolus-Tracking Studies of Human Pathology, NMR Biomed., 18:173-185 (2005), all of which are hereby incorporated by reference.

In accordance with embodiments herein, certain steps may be taken, even in the clinical setting, to improve the precision, the accuracy, and/or the diagnostic richness of the SSM DCE-MRI pharmacokinetic parameters. Such modifications may, for example, decrease the random error scatter in the FIGS. 3 and 4 point clusters. This may allow further discrimination of pathology sub-types.

The DCE-MRI time-course acquisitions discussed herein were prescribed for radiological considerations and were truncated. Increasing this period would likely improve accuracy and precision of the benign lesion parameters. For these ROIs, the maximum R₁ value is rarely reached in the no more than seven minutes usually allowed. This is the likely source of abnormally large vₑ values for some benign tumors. Increasing the period to 15 minutes may help define the shape of the time-course, even for malignant tumors.

The DCE-MRI acquisitions for the data described herein were not particularly exchange sensitive. Even so, exchange effects seem to facilitate very high discrimination of malignant from benign breast tumors.

The tissue R₁ₒ values (the pre —CR ¹H₂O longitudinal relaxation rate constants) may be mapped, and not simply assumed as they were herein. Individual AIFs may be used as well. A reference tissue method, or an automated AIF determination (e.g., Jim 4.0 software; Xinapse Systems; Thorpe Waterville, UK) may be used.

Increased temporal resolution may be achieved without sacrificing spatial resolution or signal-to-noise. Parallel RF excitation/acquisition may be useful for achieving such increased temporal resolution. With good definition of the DCE time-course first-pass leading edge, the second generation SSM (BALDERO (Blood Agent Level Dependent and Extravasation Relaxation Overview)) analysis, which accounts for blood ¹H₂O signal pharmacokinetic behavior, may be used to also determine v_{b} and k_{bo} values. It is anticipated that tumor v_{b} values will have significant diagnostic value. Furthermore, v_{b}k_{bo} is the transendothelial water permeability coefficient surface area product, P_{W}S′, where S′ is the total capillary bed surface area. The ratio P_{W}S′/P_{CR}S′ would be the intensive property P_{W}/P_{CR}. The value of the CR permeability coefficient surface area product (P_{CR}S′) may be factored from the K^{trans} parameter using the blood flow value, which may also be determined from DCE-MRI data.

The DCE-MRI pharmacokinetic images may also be spatially registered to correct for patient motion.

Image acquisition without —¹H₂C— suppression may yield signal intensities much more amenable to precision parametric mapping. The maps require sufficient acquisition contrast-to-noise ratio because pixel-by-pixel analytical modeling is more susceptible to noise. However, care must be taken to avoid contamination of ¹H₂O by unsuppressed —¹H₂C—.

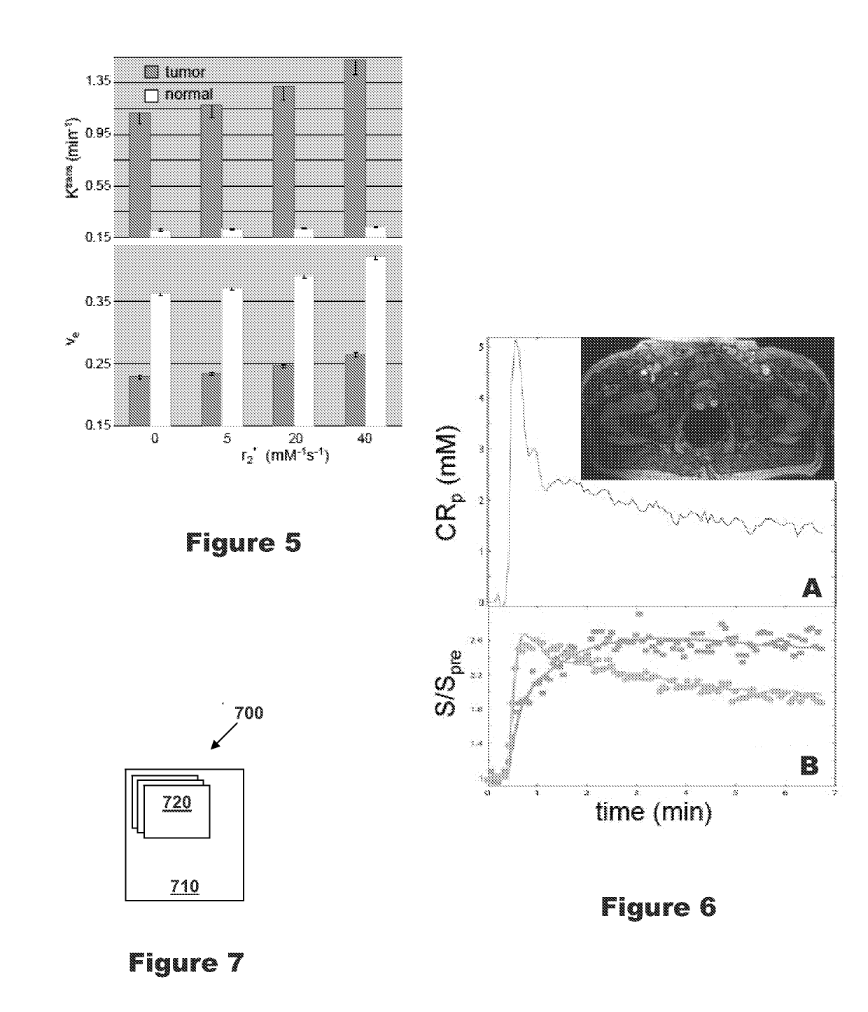

In various embodiments, the shutter-speed model may be enhanced by adding a factor for putative T₂* (transverse relaxation) signal quenching. In some embodiments, there is provided a direct application of a T₂* reduction factor to the interstitial water signal in the Ernstian MR steady-state DCE-MRI model expression. Assuming the greatest T₂* reduction will return K^{trans} and vₑ values for the tumor region of interest about 35% and 15% greater, respectively, than one would find when ignoring this effect. For normal-appearing tissues, these are 11% and 17% greater, respectively. Thus, applying the factor further distinguishes normal tissue from the tumor ROI. FIG. 5 illustrates this relationship.

The SXR-a SSM includes T₂* neglect and therefore underestimates K^{trans} and vₑ to the extent that there is a disproportionate relaxation of compartmental water signals. Embodiments herein provide a way of testing to see if the blood and interstitial water signals have been edited from the detected signal (that is, SXR-a is inappropriate).

DCE-MRI pharmacokinetic modeling usually ignores potential ¹H₂O signal reduction due to transverse relaxation (T₂*) effects. Most clinical DCE-MRI applications employ a contrast reagent (CR) dose of 0.1 mmol/kg which may produce a blood plasma CR concentration above 5.0 mM at its peak during the bolus passage. Here, using exemplary prostate DCE-MRI data, a potential T₂* effect on DCE-MRI model parameter values is described, by using a water exchange (“shutter-speed”) model along with a simplified factor to account for putative T₂* signal quenching.

Prostate ¹H₂O MRI data were acquired with a Siemens TIM Trio (3T) system under an IRB approved protocol. RF transmitting was through the whole body coil and RF receiving was with a combination of Spine Matrix and flexible Body Matrix RF coils. The DCE-MRI sequence employed a 3D TurboFLASH sequence with a 256*144*16 matrix size and a 360*203 mm² field of view, resulting in an in-plane resolution of 1.4*1.4 mm². Other parameters are: slice thickness: 3 mm; TR/TE/FA: 5.42 ms/1.56 ms/15°, imaging intersampling interval: 4.16 seconds. Any T₂*-induced signal reduction is assumed to be proportional to [exp(−(r₂*(CR)+R₂₀)·TE)], applying to the ¹H₂O signal from the CR-occupied compartment.

For the data here, the most influential CR-containing compartment is the prostate interstitium. Thus, r₂* and CR represent the interstitial CR transverse relaxivity and concentration, respectively. Since susceptibility effects cross compartmental boundaries, surely r₂* also has a contribution from capillary blood plasma CR. This T₂*-reduction factor is then directly applied to the interstitial ¹H₂O signal in the Ernstian MR steady-state DCE-MRI model expression. Parameter uncertainties were determined with sets of Monte Carlo simulations carried out for each ROI-averaged ¹H₂O signal with increasing T₂* quenching accounted for by choosing an increasing r₂* value (mM⁻¹s⁻¹): 0 (no quenching), 5 (a literature value), 20 (an estimated blood plasma value at 3T), or 40. For each r₂* and each ROI data set, 200 simulation runs were performed with Gaussian noise (μ=0, σ=0.08) directly added to the normalized ROI data time-course. This resulted in a simulated time-course with a signal-to-noise ratio (SNR) slightly better than that from a single pixel. Random initial guess values were evenly distributed within the parameter space for each simulation fitting.

FIG. 6, Panel A (inset) shows a transverse pelvic DCE image slice (anterior up/inferior perspective, approximately 34 seconds post CR injection) of a research subject. Two ROIs are indicated within the prostate gland: one in an area of retrospectively-confirmed prostate cancer, left; and the other in contralateral normal-appearing prostate tissue, right. Panel A plots the arterial input function obtained from an ROI in a femoral artery. Its magnitude was adjusted using a custom-written numerical approach and an obturator muscle ROI for reference tissue. The time-course from the first-pass (includes the initial peak) was used to estimate blood volume fraction. Color-matched tissue data time-courses (points) and representative fittings (curves) are seen in Panel B.

FIG. 5 shows how the K^{trans} (volume fraction CR transfer rate constant product, top) and vₑ (extracellular, extravascular space, EES, volume fraction, bottom) fitting results would change if increasing interstitial ¹H₂O T₂* quenching is assumed. With K^{trans} values this large, the algorithm is effectively a two-site (interstitium/cytoplasmae) exchange model, and the T₂*-induced signal reduction is applied to only the EES signal. As noted above, assuming the greatest T₂* reduction (r₂*=40 mM⁻¹s⁻¹) will return K^{trans} and vₑ values for the tumor ROI about 35% and 15% greater, respectively, than one would find ignoring this effect. For the normal-appearing tissue, these are 11% and 17% greater, respectively. Conversely, the usual literature analysis includes transverse relaxation neglect (by effectively assuming r₂*=0) and thus underestimates K^{trans} and vₑ to the extent that there is disproportionate relaxation of compartmental ¹H₂O signals.

The analysis used here is based on an inherently three-site model, but multi-step recursive fittings would eventually return a zero (within error) blood volume fraction (v_{b}) for the tumor tissue. This is not because v_{b} is actually zero, but only because it is indeterminate due to the very CR-permeable capillary wall. The blood ¹H₂O signal makes a contribution indistinguishable from that of the EES. Thus, it may be better to use an only two-site model. For consistency, the same two-site model is also used for the normal appearing tissue ROI. The current analysis is conservative in estimating EES signal T₂*-quenching effects. Interestingly, however, the extracted parameters move exactly in the direction seen comparing analyses with the fast-exchange-regime (FXR)-allowed two-site shutter-speed model with the slow-exchange-regime (SXR)-allowed version. The former neglects a distinguishable interstitial ¹H₂O signal contribution, which is reduced by exchange and may also be at least partially T₂*-quenched. For a tumor blood volume estimation using DCE-MRI with extravasating CR, it is prudent to use a lower CR dose.

Any one or more of various embodiments previously discussed may be incorporated, in part or in whole, into a computing device or a system. A suitable computing device may include one or more processors for obtaining/receiving data, processing data, etc. One or more of the processors may be adapted to perform methods in accordance with various methods as disclosed herein. A computing device may also include one or more computer readable storage media.

Any one or more of various embodiments as previously discussed or discussed below may be incorporated, in part or in whole, into an article of manufacture. In various embodiments and as shown in FIG. 7, an article of manufacture 700 may comprise a computer readable medium 710 (a hard disk, floppy disk, compact disk, etc.) and a plurality of programming instructions 720 stored in computer readable medium 710. In various ones of these embodiments, programming instructions 720 may be adapted to program an apparatus, such as an MRI device or a processor within or separate from an MRI device, to enable the apparatus to perform one or more of the previously-discussed.

Additional embodiments encompass the ability to track cancer growth and demise, as well as can predict cancer therapy response. Such embodiments can allow for MRI detection of two of the major phenotypic properties of cancers, an angiogenic switch and a metabolic switch, which combined can be a very powerful combination. Detecting tumorigenic transformation crucial to cell metastatic potential can be very valuable for therapeutic monitoring.

In addition to the conventional K^{trans} and vₑ (interstitial volume fraction) parameters, an SSM fitting of DCE-time course data can also return a third parameter, the mean intracellular water molecule lifetime, τᵢ, which accounts for the transcytolemmal exchange effects. A recent yeast cell suspension study (Zhang et al. Biophys J 101:000-000 (2011), hereby incorporated by reference herein, showed that, τᵢ, is inversely correlation with cell membrane ion ATPase kinetics, a measure of metabolism. As measures for both tumor metabolism and perfusion/permeability, τᵢ can be a sensitive DCE-MRI biomarker for evaluation of cancer (e.g., breast cancer) therapeutic response.

157 patients with 172 suspicious breast lesions [89 patients with 92 lesions at institution A (IA); 68 patients with 80 lesions at institution B (IB)] consented to research DCE-MRI studies prior to standard care biopsy procedures. The 92 lesions at IA were mammographically negative, but referred for biopsies following clinical MRI diagnoses. The 80 lesions at IB were referred for biopsies following positive mammography and/or ultrasound diagnoses. The research DCE-MRI acquisitions were performed using 1.5T GE (IA) and 3T Siemens (IB) instruments with the body transmit and 4- or 7-channel phased-array bilateral breast receive RF coils. A 3D spoiled gradient-recalled-echo (GRE) sequence was used to acquire unilateral sagittal 3 mm-thick DCEMRI images for all 89 IA and 14 IB patients, covering the breast with the suspicious lesion(s). A GRE-based 3D TWIST sequence was used to acquire bilateral axial 1.4 mm-thick DCE images from the other 54 IB patients. 10 degree flip angle and a parallel imaging acceleration factor of two were used in both sequences, with 2.2-4.2 ms TE and 5.6-7.4 ms TR for the former, and 2.9 ms TE and 6.2 ms TR for the latter. The unilateral acquisitions had a range of temporal resolution from 13 to 41 s (median: 25 s). TWIST is a k-space undersampling and data sharing GRE sequence delivering bilateral high patial resolution breast DCE-MRI at uniform 18 s temporal resolution. The DCE-MRI acquisition time was ˜8 (IA) or ˜10 (IB) min with gadolinium CA (Magnevist® at IA and Prohance® at IB) IV injection through an antecubital vein (0.1 mmol/kg at 2 mL/s) carried out following acquisition of one (IA) or two (IB) baseline image volumes. The lesion ROI and pixel-by-pixel (within ROI) DCE time-course data were subjected to both the SM and the FXR-a (fast exchange-regime-allowed) version SSM pharmacokinetic analyses to extract K^{trans}, vₑ, kₑₚ (=K^{trans}/vₑ unidirectional CA intravasation rate constant) and τᵢ (SSM only) parameters.

Receiver Operating Characteristic (ROC) curve analyses were conducted to assess the diagnostic accuracies of the DCE-MRI biomarkers, while Spearman's correlation analyses were performed to evaluate relationships between τi and other biomarkers. Biopsy pathology analyses revealed that 46 of the 172 lesions were malignant. Table 2 below lists the mean±SD lesion ROI DCE-MRI biomarker values for the malignant and benign lesions, as well as the ROC area under the curve (AUC) values with unity indicating perfect diagnostic accuracy.

TABLE 2
Table Breast lesion ROI DCE-MRI parameter and corresponding ROC AUC values

| | K^{trans} (min⁻¹) | | vₑ | | kₑₚ (min⁻¹) | | τᵢ (s) |
|---|---|---|---|---|---|---|---|
| | SM | SSM | SM | SSM | SM | SSM | SSM |
| M (N = 4.6) | 0.12 ± 0.06 | 0.21 ± 0.14 | 0.36 ± 0.17 | 0.61 ± 0.17 | 0.36 ± 0.16 | 0.34 ± 0.24 | 0.49 ± 0.22 |
| B (N = 126) | 0.050 ± 0.029^{a} | 0.056 ± 0.036^{b} | 0.41 ± 0.22 | 0.64 ± 0.18 | 0.14 ± 0.08^{c} | 0.094 ± 0.060^{d} | 0.60 ± 0.39^{e} |
| ROC AUC | 0.89 | 0.93 | 0.38 | 0.43 | 0.91 | 0.94 | 0.44 |

Mean ± SD;
M: malignant,
B: benign; unpaired t test (M vs B);
^{a}p < 0.0001;
^{b}p < 0.0001;
^{c}p < 0.0001;
^{d}p < 0.00011;
^{e}p = 0.02

For both SM and SSM analyses, the malignant lesion group has significantly (P<0.0001) higher K^{trans} and kₑₚ values than the benign group, while the SSM-only τi biomarker is significantly (P=0.02) smaller for the malignant group compared to the benign group. There is no statistically significant difference in SM or SSM vₑ values between the two groups. Based on ROC AUC values, K^{trans} and kₑₚ obtained from either model are good diagnostic markers with the SSM parameters having higher diagnostic accuracies than their SM counterparts. The difference in ROC AUC between SSM and SM K^{trans} is statistically significant (P=0.0013, nonparametric test). The vₑ and τᵢ, parameters show to be poor diagnostic markers.

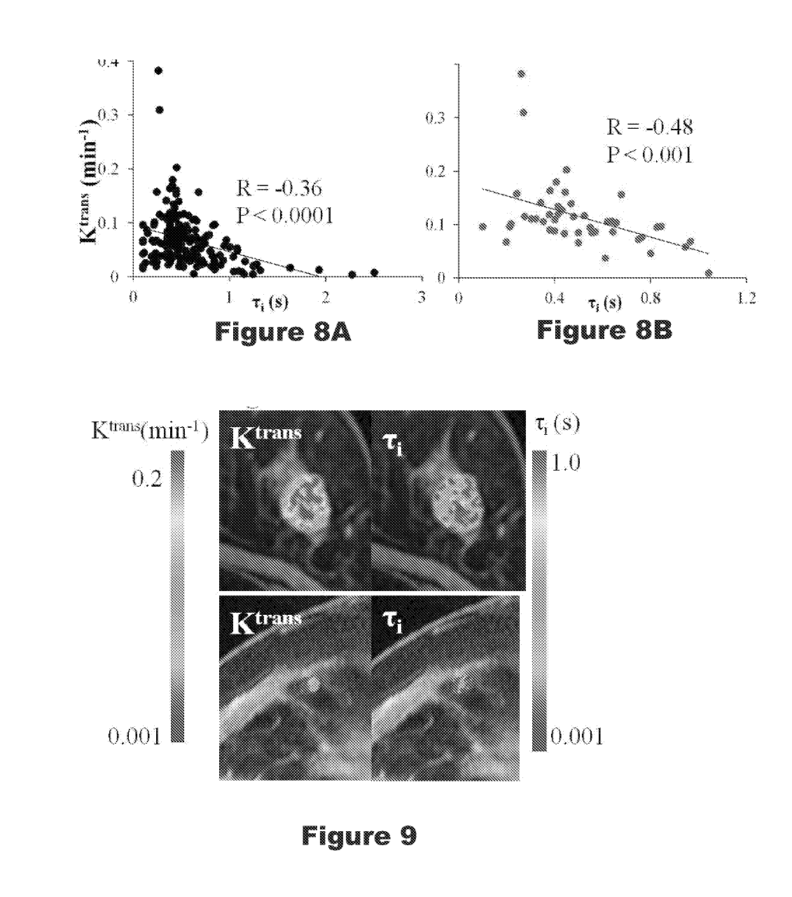

FIG. 8 shows significant inverse correlations between SM K^{trans} and τᵢ parameters for the entire lesion population (FIG. 8A) and the malignant lesions (FIG. 8B). Similar significant correlation (R=−0.16, P<0.04) was also found between SSM K^{trans} and τᵢ for the entire population. Larger Ktrans values are associated with smaller τᵢ values. This can also often be seen within lesions, as the displacement of hot spots in parametric Ktrans and τᵢ maps.

FIG. 9 shows parametric K^{trans} and τᵢ maps wherein color hot spot maps are overlaid on DCE-MRI images from a malignant (top) and a benign (bottom) lesion. In both tumors, areas with “hot” K^{trans} color generally have “cold” τᵢ color, and vice versa. Consistent with previous studies of smaller cohorts, substantial SM underestimation (relative to SSM) of K^{trans} occurred in only malignant lesions in this larger population. Since the FXL condition assumes τᵢ>0, the fact that malignant lesions have smaller τᵢ values than benign lesions shows that the greater increase of malignant lesion K^{trans} value by the SSM is not simply because it includes an additional variable (τᵢ), but because of genuine exchange effects. The significant K^{trans}/τᵢ correlation is not due to intra-model parameter co-variance because τᵢ is a SSM-only parameter and it correlates with SM K^{trans}. As mentioned earlier, τᵢ has been shown to be inversely correlated with cellular metabolic activity. The smaller τᵢ values for malignant lesions shows that they are more metabolically active (as expected), but the τᵢ hot spots areas within a malignant lesion indicate regions of hypoxia/necrosis. This can therefore be of tremendous clinical utility. K^{trans} has previously been shown to be a useful biomarker for prediction of breast cancer therapy response. The slope of the K^{trans}/τᵢ linear regress ion is −0.13 for the malignant lesions (right side of FIG. 8), which indicates that a small, therapy-induced K^{trans} change can be reflected by a larger τᵢ change, making τᵢ a sensitive DCE-MRI biomarker for evaluation of cancer therapeutic response.

Other embodiments disclosed herein may be used to separate perfused (oxygenated), hypoxic (viable), and necrotic tumor regions, thereby providing tremendous value for treatment since hypoxic cancer cells are more resistant to treatment and thus, tumor hypoxia has been related to treatment outcome and patient survival. Hypoxia imaging and the identification of tumor necrosis early after the start of treatment facilitate the assessment of treatment response before tumor shrinkage occurs. Previous techniques utilizing DCE-MRI have been able to distinguish well-perfused from necrotic tumor tissue, while identification of hypoxic regions still required additional testing using F-Fmiso PET.

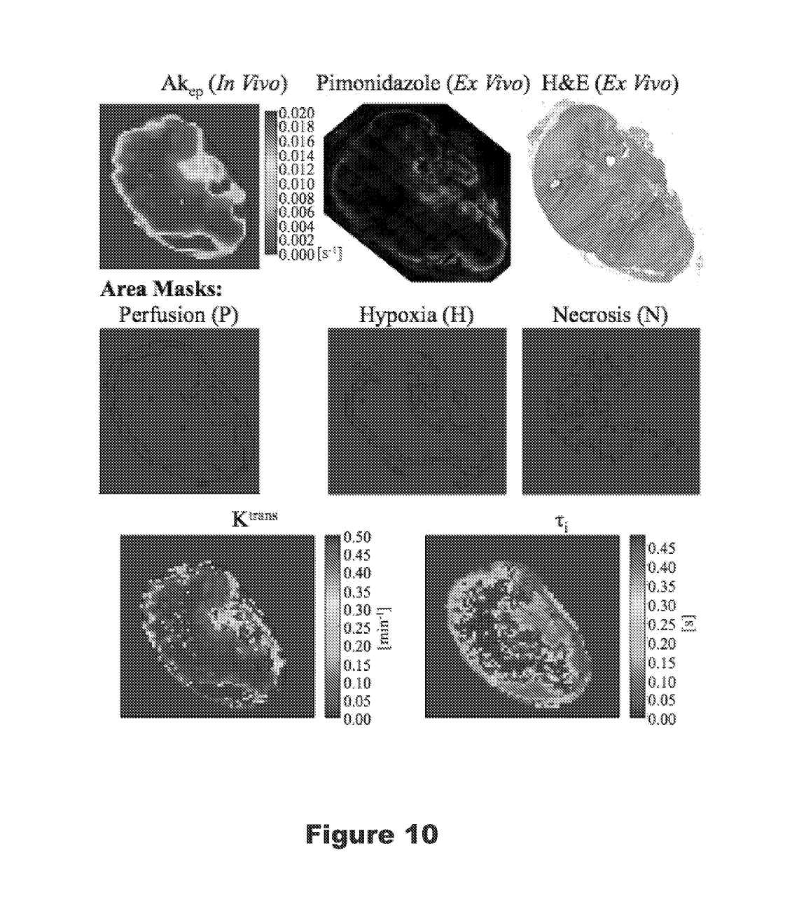

Experimental data was acquired as described in Cho H et al. Neoplasia 2009. 11(3):247, which is hereby incorporated by reference. The DCE-MRI signal intensity time-course data in the tumor region of interest (ROI) underwent pixel-by-pixel SSM pharmacokinetic analysis as described in Huang W et al. PNAS 2008. 105(46):17943 and Li X et al. Magn Reson Med 2005. 53(3):724, both of which are hereby incorporated by reference herein. The pre-contrast T1 value was calculated by comparing the signal intensity of the DCE-MRI with that of the proton density MR images acquired before contrast agent injection. The arterial input function (AIF) curve shape was taken from a direct measurement in another DCE-MRI study (Li X et al. J Magn Reson 2010. 206:190, also incorporated by reference herein) and temporally resampled to match the current DCE-MRI data. The AIF amplitude was then adjusted using a muscle ROI within the image field-of-view as reference tissue. To demonstrate the relationship of the SSM parameters K^{trans} and τᵢ with the tumor microenvironment, masks selecting pixels in the tumor that are predominantly well perfused (P), hypoxic (H), or necrotic (N) were obtained by thresholding of in vivo Aₖₑₚ maps (P), ex vivo pimonidazole (H) and Hematoxylin &Eosin (N) staining of tissue sections as shown in FIG. 10.

In more detail, FIG. 10 shows spatial, heterogeneous distribution of tumor perfusion/permeability (P), hypoxia (H) and necrosis (N) of a representative tumor slice from an experimental tumor (V=1230 mm³), including K^{trans} and τᵢ maps of the tumor slice obtained from SSM analysis (bottom row after area masks obtained by ‘thresholding’ are applied). Qualitatively, K^{trans} (lower panel) and Aₖₑₚ (top panel) show to be similarly spatially distributed and positively related, while high τᵢ values (bottom panel) show to correspond to tumor necrosis (N).

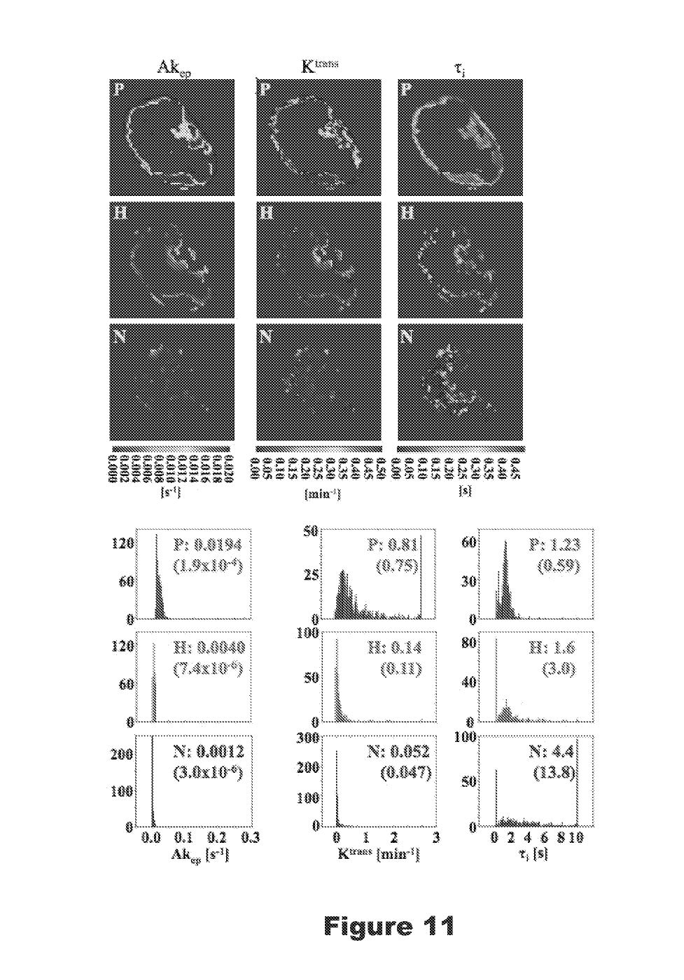

FIG. 11 depicts the spatial distribution of DCE-MRI parameters in well-perfused, hypoxic and necrotic areas of a tumor, including corresponding histograms of the regions with mean (variance) demoted. Quantitatively, perfused areas (P) are characterized by high Aₖₑₚ (see left-most columns of FIG. 11) or high K^{trans} and low τᵢ values (see two right-most columns of FIG. 11), while necrotic areas (N) are characterized by low Aₖₑₚ (see left-most column of FIG. 11A) or low K^{trans} and high τᵢ values (see two right-most columns of FIG. 11B). Hypoxic areas (H) also have low Aₖₑₚ or K^{trans}, and thus, cannot be separated from necrotic areas (N), using either parameter alone. However, the combination of K^{trans} and τᵢ can be used to separate viable/hypoxic from necrotic or viable/well-perfused areas as K^{trans} values are low and τᵢ values cover an intermediate range in hypoxic area (see histograms of FIG. 11). Overlapping values, as seen in the histograms of FIG. 11 are to be expected due to volume averaging, especially for pixels containing hypoxic cells located close to well-perfused areas. These, in fact, can be indicative of pixels containing more than one tumor characteristic. For the purposes of predicting and monitoring cancer treatment response in the clinic, results such as those described above show that the successful implementation of SSM analysis of DCE-MRI data can obviate the need for additional imaging studies, such as F-Fmiso PET, to assess tumor microenvironment.

An additional embodiment of the SSM DCE-MRI metabolic activity metric parameter τᵢ is its use as a complimentary biomarker to FDG PET in treatment staging. Breast cancer response to neoadjuvant chemotherapy (NACT) was studied utilizing SSM DCE-MRI showing breast tumor functional changes in vascular properties precede size changes in response to NACT. Neoadjuvant chemotherapy (NACT) is increasingly used before surgery to treat locally advanced breast cancer. Though pathological response is a good indicator of survival, it can be determined only after surgery. Thus, there is genuine need of noninvasive imaging method to monitor and provide early prediction of therapeutic response. This allows swift introduction of alternative treatment for non-responding patients. In addition, accurate assessment of residual disease following NACT completion improves surgery decision making such as lumpectomy vs. mastectomy. Conventionally, tumor size measurement is used to evaluate response. However, changes in tumor size often occur late during treatment and may over- or under-estimate residual disease. By measuring tumor functional changes in vascular properties, quantitative dynamic contrast-enhanced (DCE) MRI has been shown to be effective in early prediction of breast cancer response to NACT.

Three women who were diagnosed with breast cancer had primary tumor MRI size (in the longest dimension) of 1.4, 1.5, and 3.9 cm, respectively. They all underwent six cycles of NACT (with 3-week interval) as standard care before surgery. The actual NACT regimens were dependent on HER-2 receptor status (two positive and one negative). The patients consented to research DCE-MRI studies, which were performed at time-point zero (TP₀), before NACT, at TP₁, after first NACT cycle, at TP₂—after three NACT cycles, and at TP₃, after NACT completion, but before surgery. Axial bilateral DCE-MRI images with fat-saturation and full breast coverage were acquired with a 3D TWIST (Time-resolved angiography With Stochastic Trajectories) sequence using a 3T Siemens scanner.

The TWIST sequence is a k-space undersampling and data sharing gradient-echo sequence delivering both high spatial and temporal resolution for breast DCE-MRI. Other details of DCE-MRI acquisition included 10 degree flip angle, 2.9/6.2 ms TE/TR, a parallel imaging acceleration factor of two, 30-34 cm FOV, 320×320 matrix size, and 1.4 mm slice thickness. The total acquisition time was approximately 10 minutes with 18 seconds temporal resolution. Gd contrast agent (Prohance®) IV injection (0.1 mmol/kg at 2 mL/s) was carried out following acquisition of two baseline image volumes. Tumor ROIs were drawn by experienced radiologists who also measured tumor size according to well established (one dimensional) RECIST guidelines. The ROI and pixel-by-pixel (within ROI) DCE time-course data were subjected to both the SM and the SSM pharmacokinetic analyses to extract K^{trans}, vₑ, kₑₚ (=K^{trans}/vₑ), and τᵢ (from SSM only) parameters, as previously described. The whole tumor ROI DCE-MRI parameter values were calculated by averaging the ROI values from each of the image slices covering the entire tumor, weighted by the pixel numbers within the ROI in each image slice. As previously described above, novel imaging biomarkers such as ΔK^{trans}, defined as [K^{trans}(SSM)−K^{trans}(SM)], can be calculated. ΔK^{trans} is a measure of the exchange effects on K^{trans} quantification.

The pre-therapy biopsy specimens along with the post-therapy surgical specimens and lymph nodes were analyzed to evaluate pathological responses. Two pathological metrics, RCTD (relative changes in tumor density) and RCB (residual cancer burden), were computed. Pathologic complete response (pCR) is defined as RCTD=−1.0 and RCB=0; pathologic non-response (pNR) as RCTD≧0; and pathologic partial response (pPR) as −1.0<RCTD<0, which can be further stratified by RCB values: the higher the value, the more severe the residual disease.

Pathological analyses of specimens following definitive surgeries revealed that one patient had pCR (RCTD=−1.0 and RCB=0) and the other two had pPR [(RCTD=−0.66 and RCB=1.35) and (RCTD=−0.62 and RCB=3.36), respectively]. None of the MRI metrics at baseline (pre-NACT), RECIST or DCE-MRI parameters, appeared to be initially predictive of pathological response. However, the % changes in DCE-MRI biomarkers K^{trans}, kₑₚ. ΔK^{trans}, Δkₑₚ, and τᵢ after the first NACT cycle (at TP₁) were able to discriminate the pCR from the two pPRs.

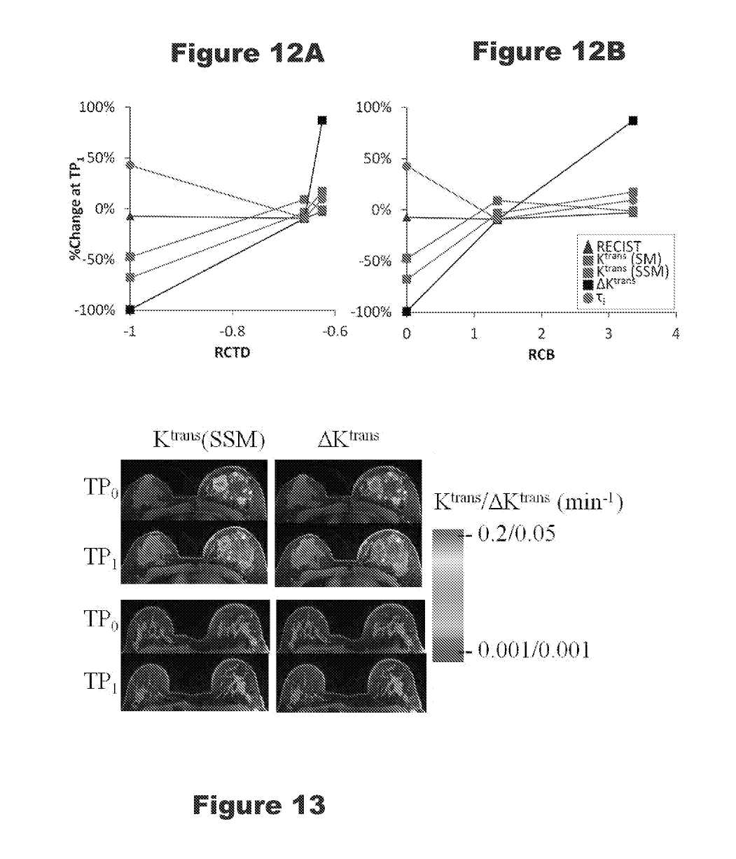

FIG. 12 shows examples of tumor region of interest (ROI) parameters K^{trans}, ΔK^{trans}, and τᵢ% changes plotted against relative changes in tumor density (RCTD) (FIG. 12A) and residual cancer burden (RCB) (FIG. 12B) values for three patients receiving neoadjuvant chemotherapy. The DEC-MRI parameter changes for the pCR were substantially larger than those of the two pPRs. The RECIST measurement, however, was not a predictor of response at this early stage of the treatment, nor was it at TP₂⁻ midpoint of treatment (not shown in FIG. 12). At TP₁, the differences in ΔK^{trans} changes were so significant that even the two pPRs were differentiated from each other, e.g., ΔK^{trans} changes at TP₁ predicted RCB after the completion of NACT.

FIG. 13 shows tumor SSM K^{trans} and ΔK^{trans} maps of a pathologic partial response (pPR) to chemotherapy (at time points TP₀ and TP₁) with RCB=3.36 (top portion of FIG. 13) and a pathologic complete response (pCR) to chemotherapy (bottom portion of FIG. 13). The decreases in both SSM K^{trans} and ΔK^{trans} from time points TP₀ to TP₁ were dramatic for the pCR.

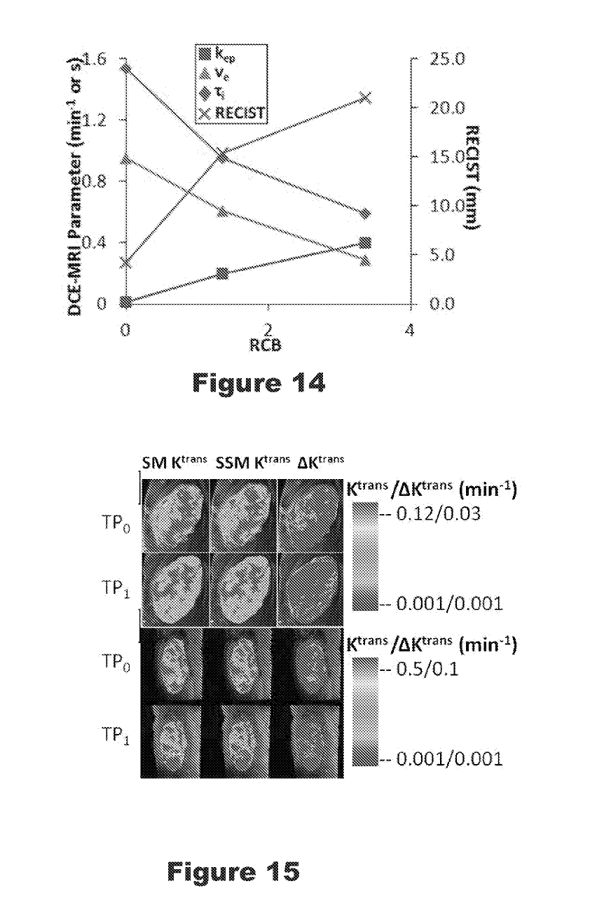

FIG. 14 shows DCE-MRI parameters after completion of neoadjuvant chemotherapy (at time point TP₃) that correlate with residual cancer burdern (RCB) and can therefore be indicators of residual disease. These included RECIST tumor size and SSM kₑₚ, vₑ, and τᵢ. Higher RCB was associated with larger tumor size, greater kₑₚ, smaller vₑ, and shorter τᵢ.

Multiple DCE-MRI biomarkers can be used for early prediction of response, though the ΔK^{trans} parameter appears to be the most sensitive (as shown herein), implying that the exchange effects on DCE-MRI pharmacokinetic data analysis may be a more responsive measure than K^{trans} or kₑₚ in assessing anti-vascular effects of the treatment. The two pPRs had RCB scores of I and III, corresponding to RCB=1.35 and 3.36, respectively. The patient with the higher RCB score after NACT could presumably have benefited from alternative treatment regimen in the early stage. Only the changes in ΔK^{trans} clearly identified this patient after the first NACT cycle. Another interesting finding is that the τᵢ parameter after NACT completion is a good indication of RCB. As noted above, recent study showed that τᵢ is inversely correlated with cellular ATP content. The association of higher RCB with shorter τᵢ is consistent with high metabolic rate in viable tumor, therefore making τᵢ a viable biomarker for treatment staging.

Another exemplary example of the ΔK^{trans} DCE-MRI SSM parameter, in comparison to other MRI metrics, for evaluation of cancer therapy to response provides for early prediction of soft-tissue sarcoma response to anti-angiogenic therapy.

Patients with biopsy-proven, grade 2-3, deep, and >5 cm soft tissue sarcomas participated in a phase I clinical trial in which the vascular endothelial growth factor receptor (VEGFR) inhibitor, Sorafenib, was added to a preoperative chemoradiotherapy regimen. Research MRI studies were performed at time-point zero (TP₀)—before therapy, TP₁, after two weeks of Sorafenib only treatment, and TP₂, after eight more weeks of treatment with Sorafenib plus chemoradiation therapy, followed by surgery and pathology review including estimation of tumor histologic necrosis. A total of eleven patients consented to the research MRI scans, with 9 of them having at least two DCE-MRI studies (at TP₀ and TP₁). In these 9 patients, five masses were located in the thigh, two in the calf, one in the knee, and one in the shoulder.

The MRI studies were performed using a 3T Siemens instrument with the body transmit and phased-array body matrix (combined with a spine matrix) receive RF coils. Following scout and axial T2-weighted MRI, sagittal diffusion-weighted imaging (DWI) was performed (in seven of the 9 patients) using a spin-echo single-shot EPI sequence with TE/TR=104/8000 ms, 24-36 cm FOV, 5 mm slice thickness with zero gap, 192×192 matrix size, and b values of 0, 500, and 1000 s/mm² applied in three orthogonal directions.

Subsequently, a 3D RF-spoiled gradient-echo sequence was used to acquire sagittal DCE-MRI data with 10 degree flip angle, TE/TR=1.56.0 ms, and 320×160 matrix size. The image FOV, slice number, location, and thickness matched those of the DWI scan. A parallel imaging acceleration factor of 2 was used for DCE-MRI, resulting in 7-16 s temporal resolutions depending on tumor size. The total DCE acquisition time was approximately 10 minutes with Gd contrast agent (Prohance®) IV injection (0.1 mmol/kg at 2 mL/s) carried out following acquisition of five baseline image volumes. Prior to DCE-MRI, proton density images were acquired with matching spatial coordinates, for pre-contrast T1 determination.

ADC trace maps were generated with manufacturer's DWI data processing software. The DCE-MRI images were processed off-line using the SM and SSM pharmacokinetic models to fit both tumor ROI and pixel-by-pixel (within the ROI) time-course data (2-4). The arterial input functions (AIFs) used for the quantitative analyses were directly measured from ROIs placed in a femoral artery (for thigh, knee, and calf tumors) and an axillary artery (for the shoulder mass). The whole tumor ROI DWI/DCE-MRI parameter values were calculated by averaging the ROI values from each of the image slices covering the entire tumor, weighted by the pixel numbers within the image slice ROIs. Pixel parameter values were analyzed with histograms and the amplitude and median values were obtained. The post-contrast DCE images at or near signal intensity time-course maxima were used to measure tumor size according to the well-established (one dimensional) RECIST guidelines.

Pathology review of the surgical specimen revealed that three of the 9 sarcomas had optimal treatment responses to the preoperative therapy with ≧95% necrosis, while the other 6 tumors had sub-optimal responses with <95% necrosis. The baseline (TP₀) MRI metrics, including tumor size, ADC, and DCE-MRI parameter values (whole tumor ROI or histographic measures), were not predictive of response to the treatment regimen.

FIG. 15 shows SM K^{trans}, SSM K^{trans}, and ΔK^{trans} maps at two time points (before therapy (TP₀) and after two weeks of Sorafenib only treatment (TP₁) of a tumor with 95% necrosis (right thigh mass, top portion of FIG. 15) and a tumor with 50% necrosis (left thigh mass, bottom portion of FIG. 15) (both necrosis levels determined at a third time point (TP₂)-8 weeks after TP₂, during which time Sorafenib plus chemotherapy was administered).) The optimal responder mass (top portion of FIG. 15) had considerable decrease in each of the three markers (SM K^{trans}, SSM K^{trans}, and ΔK^{trans}) at TP₁ with changes in ΔK^{trans} being the most dramatic, while no substantial K^{trans}/ΔK^{trans} changes were observed in the sub-optimal responder tumor (bottom portion of FIG. 15).

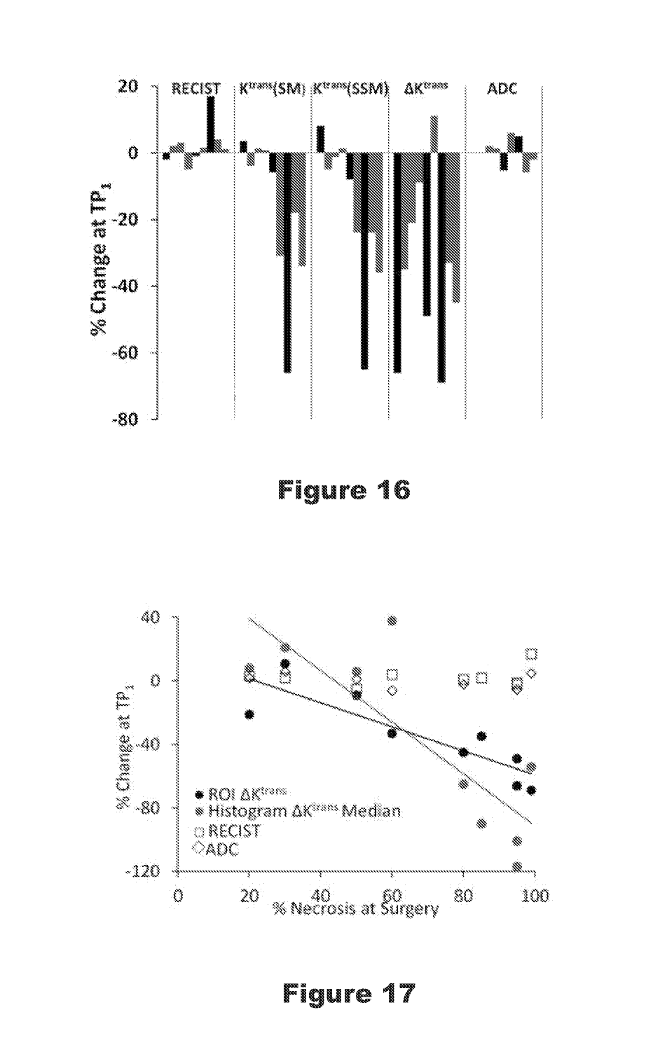

FIG. 16 shows a column graph of whole tumor region of interest (ROI) MRI biomarker (parameter) % changes after two weeks of Sorafenib only treatment (TP₁) relative to before therapy (TP₀). The black columns represent 3 optimal responders, and the gray columns the other 6 sub-optimal responders. The changes in tumor size (RECIST) and ADC were small and indiscriminate for the two groups of responders. Among ROI K^{trans}(SM),K^{trans}(SSM), and ΔK^{trans}, only % change in ΔK^{trans} was able to completely separate the optimal and sub-optimal responders. Six of the 9 patients (two optimal and four sub-optimal responders) had MRI at TP₂. Though the changes in RECIST and ADC were much smaller compared to those in K^{trans} and ΔK^{trans} at TP₂ (relative to TP₀), all MRI metrics provided discrimination of optimal/sub-optimal responders after completion of the entire treatment course (not shown visually). However, it is the 2 week TP₁ biomarker changes that are crucial for early prediction and represent the effect of the VEGFR inhibitor alone.

FIG. 17 show a scatter plot of % changes after two weeks of Sorafenib only treatment (TP₁) in RECIST (tumor size), ROI ADC (apparent diffusion coefficient), ROI ΔK^{trans}, and histogram median ΔK^{trans} vs. % necrosis at time of surgery (at TP₂) for all nine (9) patients. There were significant linear correlations between % necrosis and % changes in ROI ΔK^{trans} (R=−0.93, P=0.0003; Spearman's correlation), and histogram median ΔK^{trans} (R=−0.71, P=0.03). No such relationships were observed for changes in either K^{trans}(SM) or K^{trans}(SSM). Such results show that DCE-MRI biomarkers are more effective than tumor size and ADC measures for early prediction of sarcoma response to antiangiogenic treatment. Tumor vascular shut-down induced by Sorafenib preceded cell death and tumor treatment and such vascular shut-down can be detected by DCE-MRI biomarkers. Significant ADC changes were detectable only when there were substantial changes in tumor volume in a recent study using DWI to assess sarcoma response to therapy. This may also explain why ADC was not a good predictor of response to TP₁ in this exemplary study, as the tumor size changes were minuscule. ΔK^{trans} is shown to be more sensitive to therapy-induced tumor vascular changes than K^{trans} itself, and thus a good early predictor of soft-tissue sarcoma pathologic response.

Early identification of patients not responding to therapy may allow for prompt alternative treatments, sparing them from ineffective and potentially toxic therapies. As an additional benefit, the ΔK^{trans} calculation may mitigate or eliminate many common systematic DCE-MRI parameter errors, for example, from AIF uncertainty, since the SM and SSM analyses use the same AIF. Such systematic errors have long been principal challenges in using quantitative DCE-MRI for therapy monitoring.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A computer-implemented method for determining a level of cellular metabolic activity for a region of interest, the method comprising:
receiving a first set of DCE-MRI time-course data for a region, wherein a contrast reagent is administered prior to imaging;
identifying a region of interest from the first set of DCE-MRI time-course data for further analysis; and
analyzing the first set of DCE-MRI time-course data for the region of interest using computer implemented software to produce a first SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity for the region of interest.

2. The method of claim 1, wherein the region of interest is located in the breast of a human.

3. The method of claim 2, wherein the method is performed before biopsy.

4. The method of claim 1, where identifying a region of interest for further analysis is done manually.

5. The method of claim 1, where the identifying a region of interest for further analysis is done automatically.

6. The method of claim 1, further comprising spacially registering the image data to correct for a patient's movement during imaging.

7. The method of claim 1, wherein the received image data is acquired using unsupressed —¹H₂C—.

8. The method of claim 1, wherein the received time-course data is taken over a period of time greater than about seven minutes.

9. The method of claim 1, further comprising:
receiving a second set of DCE-MRI time-course data for the region of interest, wherein the second set of DCE-MRI time-course data is obtained after the region has been treated;
analyzing the second set of data for the region of interest using computer implemented software to produce a second SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity; and
determining the difference between the first SSM Tᵢ value and the second SSM Tᵢ value.

10. The method of claim 9, further comprising spacially registering the second set of data to correct for a patient's movements during imaging.

11. The method of claim 9, wherein the second set of data is acquired using unsupressed —¹H₂C—.

12. The method of claim 9, wherein the second set of time-course data is taken over a period of time greater than about seven minutes.

13. A method of tissue characterization based on water kinetics, the method comprising:
receiving DCE-MRI time-course data for a region, wherein a contrast reagent is administered prior to imaging;
identifying a region of interest from the DCE-MRI time-course data for further analysis;
analyzing the DCE-MRI time-course data for the region of interest using computer implemented software to produce a SM K^{trans} value, wherein the water exchange between cells or blood and interstitial spaces is assumed to be substantially infinitely fast;
analyzing the DCE-MRI time-course data for the region of interest using computer implemented software to produce a SSM K^{trans} value, where the water exchange between cells or blood and interstitial spaces is assumed to have a finite speed resulting from interaction with the contrast reagent;
analyzing the DCE-MRI time-course data for the region of interest using computer implemented software to produce a SSM Tᵢ value that accounts for transcytolemmal exchange effects; and
plotting SM K^{trans} and SSM K^{trans} v. SSM Tᵢ to determine a value for the correlation between SM K^{trans} and SSM K^{trans} and SSM Tᵢ.

14. The method of claim 13, further comprising determining a Δ K^{trans} value comprising SSM K^{trans}−SM K^{trans}.

15. The method of claim 13, wherein the region of interest is located in the heart of a human.

16. The method of claim 13, wherein the region of interest is located in the breast of a human.

17. A computer-implemented method for determining a level of cellular metabolic activity for a region of interest, the method comprising:
receiving DCE-MRI time-course data for a region, wherein a contrast reagent is administered prior to imaging;
analyzing the DCE-MRI time-course data using computer implemented software to correct for potential ¹H₂O signal reduction due to transverse relaxation effects;
identifying a region of interest from the DCE-MRI data for further analysis;
analyzing the DCE-MRI time-course data for the region of interest using computer implemented software to produce a first SSM Tᵢ value that accounts for transcytolemmal exchange effects, wherein the water exchange between cells or blood and interstitial spaces is assumed to have a finite speed resulting from interaction with the contrast reagent, and wherein Tᵢ is indicative of the level of cellular metabolic activity for the region of interest.

18. The method of claim 17, wherein the region of interest is located in the prostate of a human.

19. The method of claim 17, wherein the received image data is acquired using unsupressed —¹H₂C—.

20. The method of claim 19, wherein the received time-course data is taken over a period of time greater than about seven minutes.
